(19) United States
(12) Patent Application Publication
(10) Pub. No.: US 2024/0115748 A1
(43) Pub. Date: Apr. 11, 2024

(54) SANITIZATION OF WATER TREATMENT SYSTEM

(71) Applicant: IMPACT WATER PRODUCTS, Ontario, CA (US)

(72) Inventor: Edward Allan HORNER, Riverside, CA (US)

(21) Appl. No.: 18/482,128

(22) Filed: Oct. 6, 2023

Related U.S. Application Data
(60) Provisional application No. 63/414,153, filed on Oct. 7, 2022.

(51) Int. Cl. A61L 2/20; C02F 1/44

(52) U.S. Cl. CPC A61L 2/202; C02F 1/441; A61L 2202/11; A61L 2202/15; A61L 2202/20

(57) ABSTRACT

In some embodiments, a water ozonation system can include an ozone generator including a corona discharge unit configured to provide ozonated air as an output. The water ozonation system can further include a tubing having a first end coupled to the output of the corona discharge unit and configured to deliver the ozonated air to a second end. The water ozonation system can further include an injector assembly having a suction port coupled to the second end of the tubing and in communication with a Venturi tube having an input and an output, such that flow of liquid from the input to the output results in a suction being formed through the suction port to thereby draw the ozonated air into the liquid flowing through the Venturi tube.

### CROSS-REFERENCE TO RELATED APPLICATION(S)

This application claims priority to U.S. Provisional Application No. 63/414,153 filed Oct. 7, 2022, entitled SANITIZATION OF WATER TREATMENT SYSTEM, the disclosure of which is hereby expressly incorporated by reference herein in its respective entirety.

### BACKGROUND

### Field

The present disclosure relates to devices, systems and methods related to sanitization of water treatment systems.

### Description of the Related Art

In many water treatment systems, water can be treated by a reverse osmosis (RO) process. Such a process can be achieved to treat water from a source, and the treated water can be provided to, for example, a faucet.

### SUMMARY

In some implementations, the present disclosure relates to a water ozonation system that includes an ozone generator including a corona discharge unit configured to provide ozonated air as an output, and a tubing having a first end coupled to the output of the corona discharge unit and configured to deliver the ozonated air to a second end. The water ozonation system further includes an injector assembly having a suction port coupled to the second end of the tubing and in communication with a Venturi tube having an input and an output, such that flow of liquid from the input to the output results in a suction being formed through the suction port to thereby draw the ozonated air into the liquid flowing through the Venturi tube.

In some embodiments, the corona discharge unit can further include an input configured to allow air to be drawn in due to the ozonated air being drawn to the Venturi tube. The corona discharge unit can be configured to generate ozone through a PCS corona discharge ozone generation process.

In some embodiments, the input of the Venturi tube can be configured to be coupled to a water source, and the output of the Venturi tube can be configured to be coupled to a water treatment system. The water treatment system can include a reverse-osmosis (RO) system. The Venturi tube can be configured such that water pressure at the input is greater than water pressure at a constricted portion of the Venturi tube.

In some implementations, the present disclosure relates to a water system that includes a water treatment system configured to receive water from a source and provide treated water to a faucet, and an injector assembly including an input in communication with the source and an output in communication with the water treatment system. The injector assembly further includes a Venturi tube implemented between the input and the output, and a suction port, such that flow of water from the input to the output results in a suction being formed at the suction port. The water system further includes an ozone generator having an output in communication with the suction port of the injector assembly, such that ozonated air generated by the ozone generator is drawn into the Venturi tube by the suction and thereby introduced to the flow of water to the water treatment system.

In some embodiments, the ozone generator can include a corona discharge unit having an input configured to allow air to be drawn in due to the ozonated air being drawn to the Venturi tube by the suction. The corona discharge unit can be configured to generate ozone through a PCS corona discharge ozone generation process.

In some embodiments, the water treatment system can include a reverse-osmosis (RO) system. In some embodiments, the Venturi tube can be configured such that water pressure at the input is greater than water pressure at a constricted portion of the Venturi tube.

In some implementations, the present disclosure relates to a kit for sanitizing a water treatment system configured to receive water from a source and provide treated water. The kit includes an injector including an input configured to couple to the source and an output configured to couple to the water treatment system, a Venturi tube implemented between the input and the output, and a suction port configured to couple to a gas supply, such that flow of water from the input to the output results in a suction being formed at the suction port to thereby draw gas from the gas supply and mix at least some of the gas into the flow of water. The kit further includes an instruction for operation of the injector.

In some embodiments, the instruction can include a printed instruction. In some embodiments, the gas supply can include an ozone generator, such that the gas drawn into the Venturi tube includes ozone.

In some embodiments, the kit can further include an ozone generator, such that the gas drawn into the Venturi tube includes ozonated air. The ozone generator can include a corona discharge unit having an input configured to allow air to be drawn in due to the ozonated air being drawn to the Venturi tube by the suction.

In some embodiments, the water treatment system can include a reverse-osmosis (RO) system.

In some embodiments, the injector can be configured to be implemented with an existing reverse-osmosis (RO) system. In some embodiments, the kit can further include fittings configured to couple the injector between the source and the reverse-osmosis system. In some embodiments, at least some of the fittings can be configured to allow the injector to be removed from being coupled between the source and the reverse-osmosis system.

For purposes of summarizing the disclosure, certain aspects, advantages and novel features of the inventions have been described herein. It is to be understood that not necessarily all such advantages may be achieved in accordance with any particular embodiment of the invention. Thus, the invention may be embodied or carried out in a manner that achieves or optimizes one advantage or group of advantages as taught herein without necessarily achieving other advantages as may be taught or suggested herein.

### BRIEF DESCRIPTION OF THE DRAWINGS

FIG. 1 shows a water system having a reverse osmosis (RO) system that is configured to treat water from a source and provide the treated water to a faucet.

FIG. 2A shows an example of an ozone generator that can be included in an ozone system of the water system of FIG. 1.

FIG. 2B shows an example of a corona discharge unit 150 that can be included in the ozone generator of FIG. 2A.

FIG. 3 shows an example of an injector assembly that can be included in the water system of FIG. 1.

FIG. 4 shows that in some embodiments, one or more features of the present disclosure can be implemented as a kit.

### DETAILED DESCRIPTION OF SOME EMBODIMENTS

The headings provided herein, if any, are for convenience only and do not necessarily affect the scope or meaning of the claimed invention.

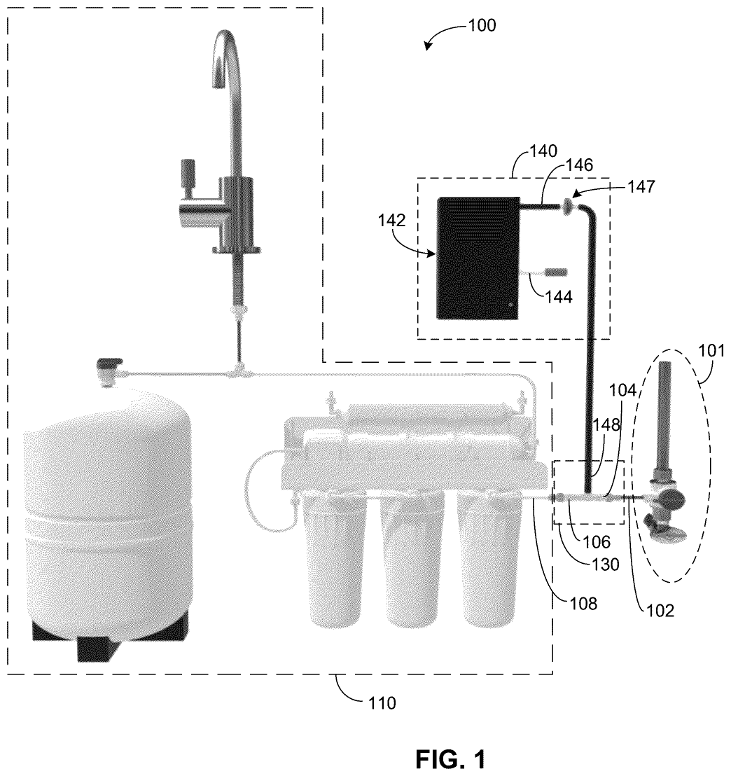

FIG. 1 shows a water system 100 having a reverse osmosis (RO) system 110 that is configured to treat water from a source 101 and provide the treated water to a faucet. More particularly, an input 108 of the reverse osmosis system 110 is shown to be coupled to the water source 101 through a tubing 102 and an injector assembly 130. Examples related to the injector assembly 130 are described herein in greater detail.

For the purpose of description, it will be understood that a tubing can be any conduit suitable for gas and/or liquid plumbing applications.

It will also be understood that while various examples are described herein in the context of a reverse osmosis system, one or more features of the present disclosure can also be utilized with other types of water treatment systems.

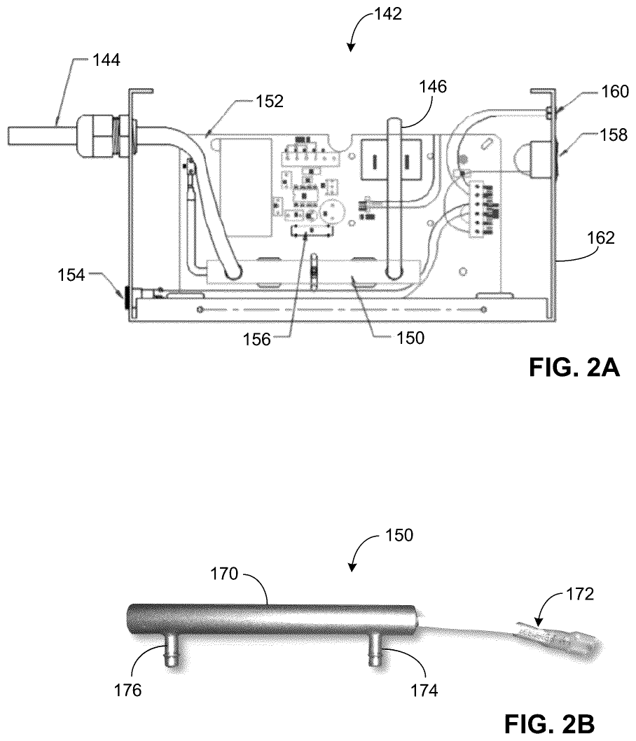

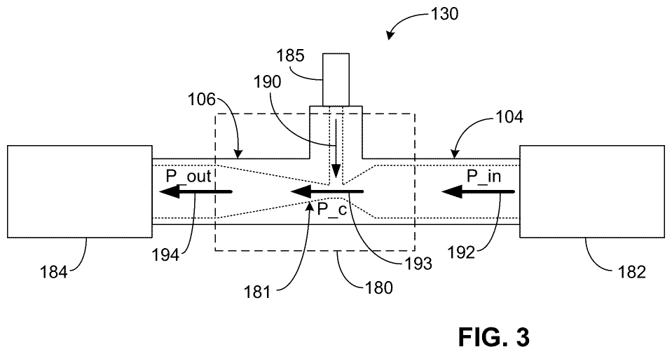

In FIG. 1, the injector assembly 130 is shown to couple the water source 101 to the reverse osmosis system 110, and also provide an input for a gas from an ozone system 140. FIGS. 2A and 2B show examples related to such an ozone system, and FIG. 3 shows an example of the injector assembly 130.

Referring to FIGS. 1, 2A and 2B, the ozone system 140 can include an ozone generator 142 having a corona discharge unit 150. In some embodiments, such a corona discharge unit can be configured to generate ozone through, for example, a PCS corona discharge ozone generation process. In such a process, a pulse modulated corona discharge generator creates ozone through an action of high voltage, low current electrical “arcs” across an air space. When oxygen (O2) is passed through the air space, some molecules are split resulting in “free” oxygen atoms which quickly attach themselves to intact O2 molecules, resulting in a very unstable form of oxygen, O3 (ozone) having superior oxidation capabilities.

In the examples of FIGS. 1, 2A and 2B, the corona discharge unit 150 is shown to include an electrical assembly 172 for providing the electrical arcs within an ozone generation chamber 170, an input 174 for providing air to the chamber 170, and an output 176 for providing ozonated air from the chamber 170. In FIGS. 1 and 2A, the input 174 (FIG. 2B) of the corona discharge unit 150 is shown to be in communication with an air-input tubing 144 for drawing in, for example, ambient air, and the output 176 (FIG. 2B) of the corona discharge unit 150 is shown to be in communication with an output tubing 146 that is in communication with a gas-input of the injector assembly 130 (e.g., through a check valve 147 and a tubing 148). In some embodiments, the check valve 147 can prevent back-flow of gas back into the corona discharge unit 150.

Referring to FIG. 2A, the ozone generator 142 is shown to further include a housing 162 that supports the corona discharge unit 150, as well as a circuit board 152 configured to provide various electrical functionalities including the corona discharge ozone generation process. Such a circuit board can also be configured to provide circuit protection utilizing, for example, a fuse 156, and to receive external power through a power input connector (e.g., for receiving DC input power).

In some embodiments, the ozone generator 142 can be configured to operate in different ozone-production modes (e.g., high and low production modes), and such modes can be controlled by a switch 158. The ozone generator 142 can also be configured to provide operating status by, for example, a status indicator 160 (e.g., power status and/or ozone-production mode status LED(s)).

It is noted that when the water system 100 as described herein is operating with the injector assembly 130 as shown in FIG. 1, flow of water from the source 101 to the RO system 110 results in suction being formed through the tubing 148. Accordingly, ozonated air is drawn from the corona discharge unit 150 through its output (176 in FIG. 2B) and the tubing (146 in FIGS. 1 and 2A). Such drawing of the ozonated air to the injector assembly 130 results in air being drawn into the corona discharge unit 150 from the air-input tubing 144 and the input 174.

FIG. 3 shows that in some embodiments, the injector assembly 130 of FIG. 1 can be configured as a Venturi injector 180 having a Venturi tube 181 that includes an input 104 and an output 106, and a constricted portion therebetween. When the Venturi tube 181 is shaped accordingly, an input flow of fluid (e.g., water) 192 has a pressure P_in that is greater than a pressure P_c of a constricted flow 193, such that a suction 190 into the Venturi tube 181 is created. Accordingly, the suction 190 can draw a gas into the Venturi tube 181 through an input 185.

In the example of FIG. 1, the foregoing input 185 (FIG. 3) is in communication to the output of the ozone generator 142 through the tubing 148. Accordingly, ozonated air from the ozone generator 142 can be introduced to the Venturi tube 181 when water is flowing through the Venturi tube 181. Thus, the ozonated air can be introduced into the water passing through the Venturi tube 181, and such ozonated water can be delivered to the reverse osmosis system 110.

In the example of FIG. 3, the input 104 of the Venturi tube 181 is shown to be coupled to a tubing 182, and the output 106 of the Venturi tube 181 is shown to be coupled to a tubing 184. In some embodiments, the tubing 182 can be one end of, or be coupled to, the tubing 102 (FIG. 1) for providing water from the source 101. Similarly, the tubing 184 can be one end of, or be coupled to, the tubing 108 (FIG. 1) for providing water to the reverse osmosis system 110.

Configured in the foregoing manner, ozone can be introduced into a reverse osmosis system for a sanitizing process. As described herein, such ozone can be produced by the ozone generator 142 (FIGS. 1 and 2A) utilizing, for example, a PCS corona discharge ozone generation process. In some embodiments, such ozone generation can produce approximately 220 mg of ozone per hour.

In some embodiments, the ozone system 140 and the injector assembly 130 can be configured to operate with an existing reverse osmosis system. In some embodiments, water flow rate of approximately 1 gallon/min (GPM) can provide sufficient suction into the Venturi tube (181 in FIG. 3) to draw and introduce ozonated air into the water.

In some embodiments, the ozone generator 142 of FIGS. 1 and 2A can be configured to operate with a transformer that converts a standard 110V AC power into a 12V DC power.

In some embodiments, the ozone system 140 and the injector assembly 130 can be installed into a reverse osmosis system periodically (e.g., once a year or as needed) for a sanitizing process. It is noted that a traditional way of sanitizing a reverse osmosis system is to use bleach or other similar chemical, and such a sanitizing process is typically difficult and time consuming. Utilizing the ozone system 140 and the injector assembly 130 as described herein can allow for use of ozone which does not need to be rinsed from the system, thereby allowing a sanitizing procedure to be performed in a chemical free and very quick manner.

By way of an example, a reverse osmosis system can be sanitized utilizing an ozone system and an injector assembly as described herein, as follows. The reverse osmosis system can be prepared by draining the tank and removing some or all associated filters and membranes. Filter sumps and membrane housing can be cleaned with soap and water. After such cleaning, the housings can be reinstalled, and any inline filters can be bypassed.

Following the foregoing cleaning process, an injector assembly as described herein can be installed between the water source and the reverse osmosis system. Water can be turned on at the source, and the faucet can be opened. Thus, water flowing through the injector assembly causes suction on the ozone injector line from the ozone generator.

Once suction is established on the ozone injector line, the ozone generator can be powered to generate ozone which is then sucked into the injector assembly. For the example ozone generation level described herein, water can be allowed to run for a selected duration (e.g., 5 minutes or more), and/or until one can detect an ozone smell in the water coming out of the faucet. Upon such completion, the faucet can be closed, and the tank can be allowed to fill for a selected duration (e.g., about 1 minute). Then, the inlet water line can be closed and the water in the reverse osmosis system can be allowed to sit for a duration (e.g., about 5 minutes).

The faucet can be opened to drain the reverse osmosis tank. If filters are to be replaced, new filters can be installed, and the reverse osmosis tank can be filled to put the reverse osmosis system back into service.

In some embodiments, an injector assembly as described herein can be installed between the water source (101 in FIG. 1) and the reverse osmosis system (110 in FIG. 1) during a sanitizing process, and be removed after the sanitizing process. For example, appropriate fittings can be provided on the tubing 102 on the source side and the tubing 108 on the reverse osmosis side to allow insertion of the injector assembly to be inline between the tubing 102 and the tubing 108 for the sanitizing process. Once such a sanitizing process is completed, the injector assembly can be removed, and an appropriate tube can be inserted in place of the injector assembly to provide a connection between the tubings 102 and 108 for normal operation of the reverse osmosis system.

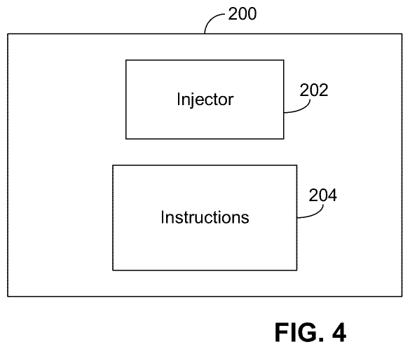

FIG. 4 shows that in some embodiments, one or more features as described herein can be included in a kit 200. For example, an injector 202 as described herein can be included in the kit 200. In some embodiments, such a kit can further include instructions 204 such as written instructions.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Description using the singular or plural number may also include the plural or singular number respectively. The word “or” in reference to a list of two or more items, that word covers all of the following interpretations of the word: any of the items in the list, all of the items in the list, and any combination of the items in the list.

The above detailed description of embodiments of the invention is not intended to be exhaustive or to limit the invention to the precise form disclosed above. While specific embodiments of, and examples for, the invention are described above for illustrative purposes, various equivalent modifications are possible within the scope of the invention, as those skilled in the relevant art will recognize. For example, while processes or blocks are presented in a given order, alternative embodiments may perform routines having steps, or employ systems having blocks, in a different order, and some processes or blocks may be deleted, moved, added, subdivided, combined, and/or modified. Each of these processes or blocks may be implemented in a variety of different ways. Also, while processes or blocks are at times shown as being performed in series, these processes or blocks may instead be performed in parallel, or may be performed at different times.

The teachings of the invention provided herein can be applied to other systems, not necessarily the system described above. The elements and acts of the various embodiments described above can be combined to provide further embodiments.

While some embodiments of the inventions have been described, these embodiments have been presented by way of example only, and are not intended to limit the scope of the disclosure. Indeed, the novel methods and systems described herein may be embodied in a variety of other forms; furthermore, various omissions, substitutions and changes in the form of the methods and systems described herein may be made without departing from the spirit of the disclosure. The accompanying claims and their equivalents are intended to cover such forms or modifications as would fall within the scope and spirit of the disclosure.

What is claimed is:

1. A water ozonation system comprising:
an ozone generator including a corona discharge unit configured to provide ozonated air as an output;
a tubing having a first end coupled to the output of the corona discharge unit and configured to deliver the ozonated air to a second end; and
an injector assembly having a suction port coupled to the second end of the tubing and in communication with a Venturi tube having an input and an output, such that flow of liquid from the input to the output results in a suction being formed through the suction port to thereby draw the ozonated air into the liquid flowing through the Venturi tube.

2. The system of claim 1, wherein the corona discharge unit further includes an input configured to allow air to be drawn in due to the ozonated air being drawn to the Venturi tube.

3. The system of claim 2, wherein the corona discharge unit is configured to generate ozone through a PCS corona discharge ozone generation process.

4. The system of claim 1, wherein the input of the Venturi tube is configured to be coupled to a water source, and the output of the Venturi tube is configured to be coupled to a water treatment system.

5. The system of claim 4, wherein the water treatment system includes a reverse-osmosis (RO) system.

6. The system of claim 4, wherein the Venturi tube is configured such that water pressure at the input is greater than water pressure at a constricted portion of the Venturi tube.

7. A water system comprising:
a water treatment system configured to receive water from a source and provide treated water to a faucet;
an injector assembly including an input in communication with the source and an output in communication with the water treatment system, a Venturi tube implemented between the input and the output, and a suction port, such that flow of water from the input to the output results in a suction being formed at the suction port; and
an ozone generator having an output in communication with the suction port of the injector assembly, such that ozonated air generated by the ozone generator is drawn into the Venturi tube by the suction and thereby introduced to the flow of water to the water treatment system.

8. The system of claim 7, wherein the ozone generator includes a corona discharge unit having an input configured to allow air to be drawn in due to the ozonated air being drawn to the Venturi tube by the suction.

9. The system of claim 8, wherein the corona discharge unit is configured to generate ozone through a PCS corona discharge ozone generation process.

10. The system of claim 7, wherein the water treatment system includes a reverse-osmosis (RO) system.

11. The system of claim 7, wherein the Venturi tube is configured such that water pressure at the input is greater than water pressure at a constricted portion of the Venturi tube.

12. A kit for sanitizing a water treatment system configured to receive water from a source and provide treated water, the kit comprising:
an injector including an input configured to couple to the source and an output configured to couple to the water treatment system, a Venturi tube implemented between the input and the output, and a suction port configured to couple to a gas supply, such that flow of water from the input to the output results in a suction being formed at the suction port to thereby draw gas from the gas supply and mix at least some of the gas into the flow of water; and
an instruction for operation of the injector.

13. The kit of claim 12, wherein the instruction includes a printed instruction.

14. The kit of claim 12, wherein the gas supply includes an ozone generator, such that the gas drawn into the Venturi tube includes ozone.

15. The kit of claim 12, further comprising an ozone generator, such that the gas drawn into the Venturi tube includes ozonated air.

16. The kit of claim 15, wherein the ozone generator includes a corona discharge unit having an input configured to allow air to be drawn in due to the ozonated air being drawn to the Venturi tube by the suction.

17. The kit of claim 12, wherein the water treatment system includes a reverse-osmosis (RO) system.

18. The kit of claim 12, wherein the injector is configured to be implemented with an existing reverse-osmosis (RO) system.

19. The kit of claim 18, further comprising fittings configured to couple the injector between the source and the reverse-osmosis system.

20. The kit of claim 19, wherein at least some of the fittings are configured to allow the injector to be removed from being coupled between the source and the reverse-osmosis system.
